(19) United States
(12) Patent Application Publication
(10) Pub. No.: US 2021/0030594 A1
(43) Pub. Date: Feb. 4, 2021

(54) PROTECTIVE SHEATH FOR EAR CANAL

(71) Applicant: Acclarent, Inc., Irvine, CA (US)

(72) Inventors: Ehsan Shameli, Irvine, CA (US); Babak Ebrahimi, Irvine, CA (US); Fatemeh Akbarian, Rancho Palos Verdes, CA (US); Elaheh Alizadeh-birjandi, Hillsboro, OR (US); Itzhak Fang, Irvine, CA (US)

(21) Appl. No.: 15/930,816

(22) Filed: May 13, 2020

Related U.S. Application Data
(60) Provisional application No. 62/879,585, filed on Jul. 29, 2019.

(51) Int. Cl. A61F 11/08; A61F 11/00; A61B 1/227; A61M 1/00; A61B 1/05

(52) U.S. Cl. CPC A61F 11/08; A61F 11/004; A61B 1/227; A61M 2210/0662; A61B 1/05; A61F 2011/085; A61F 2240/001; A61M 1/0023

(57) ABSTRACT

A method of protecting an ear canal of a patient during a surgical procedure using a protective sheath includes forming the protective sheath using additive manufacturing based on a digital model of anatomy of the patient, where the protective sheath includes a passageway extending therethrough. The method may further include inserting the protective sheath into the patient so that the passageway extends at least partially within the ear canal of the patient. The method may further include inserting at least one instrument during the surgical procedure through the passageway of the protective sheath and into the middle ear of the patient.

### PRIORITY

This application claims priority to U.S. Provisional Patent App. No. 62/879,585, entitled “Protective Sheath for Ear Canal,” filed Jul. 29, 2019, the disclosure of which is incorporated by reference herein.

### BACKGROUND

Referring to FIG. 1, the ear (10) is divided into three parts: an external ear (12), a middle ear (14) and an inner ear (16). The external ear (12) consists of an auricle (18) and the ear canal (20) that gather sound and direct it toward an eardrum (22) (also referred to as the tympanic membrane) located at an inner end (24) of the ear canal (20). The middle ear (14) lies between the external and inner ears (12, 16) and is connected to the back of the throat by a Eustachian tube (ET) (26), which serves as a pressure equalizing valve between the ear (10) and the sinuses. The ET (26) terminates in a pharyngeal ostium (28) in the nasopharynx region (30) of the throat (32). In addition to the eardrum (22), the middle ear (14) also consists of three small ear bones (ossicles): the malleus (34) (hammer), the incus (36) (anvil) and the stapes (38) (stirrup). These bones (34, 36, 38) transmit sound vibrations to the inner ear (16) and thereby act as a transformer, converting sound vibrations in the ear canal (20) of the external ear (12) into fluid waves in the inner ear (16). These fluid waves stimulate several nerve endings (40) that, in turn, transmit sound energy to the brain where it is interpreted.

Methods for treating the middle ear (14) and the ET (26) include those disclosed in U.S. Patent Pub. No. 2010/0274188, entitled “Method and System for Treating Target Tissue within the ET,” published on Oct. 28, 2010, the disclosure of which is incorporated by reference herein; U.S. Patent Pub. No. 2013/0274715, entitled “Method and System for Eustachian Tube Dilation,” published on Oct. 17, 2013, the disclosure of which is incorporated by reference herein; and U.S. patent application Ser. No. 14/317,269, entitled “Vent Cap for a Eustachian Tube Dilation System,” filed Jun. 27, 2014, the disclosure of which is incorporated by reference herein. As described in those references, functioning of the ET (26) may be improved by dilating the ET (26) with an expandable dilator instrument.

One of the approaches to a middle ear (14) surgery is the transcanal approach, during which the eardrum (22) is endoscopically visualized and accessed via the ear canal (20). Instrumentation disposed in the ear canal (20) may be utilized to traverse the eardrum (22) and operate within the middle ear (14). One of the side-effects to the transcanal approach is that the instruments (e.g. surgical tools) may cause a disturbance to the lining and tissue of the ear canal (20) due to frequent insertion and extraction of instruments into the ear canal (20). The disturbance to the healthy tissue of the ear canal (20) may be exacerbated by the tight and twisted profile of the ear canal (20). As a result, it is desirable to limit this disturbance when perfuming a middle ear (14) surgery using the transcanal approach.

While a variety of surgical instruments and methods have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a cross-sectional view of a human ear showing the inner, middle, and outer ear portions and the Eustachian tube connecting the middle ear with the nasopharynx region of the throat;

FIG. 2 depicts a perspective view of an exemplary ear canal protective system that includes an exemplary protective sheath that is coupled with a camera assembly, a suction source, and a fluid source;

FIG. 3 depicts a schematic cross-sectional view of the protective sheath of FIG. 2, taken along line 3-3 of FIG. 2, where the protective sheath is coupled with the camera assembly, the suction source, and the fluid source, and instruments extend through a passageway of the protective sheath;

FIG. 3A depicts a schematic cross-sectional view of an exemplary alternative protective sheath similar to the protective sheath of FIG. 3;

FIG. 4 depicts a first exemplary method of protecting an ear canal of a patient using the protective sheaths of FIGS. 2-3A; and

FIG. 5 depicts a second exemplary method of protecting an ear canal of a patient using the protective sheaths of FIG. 2-3A.

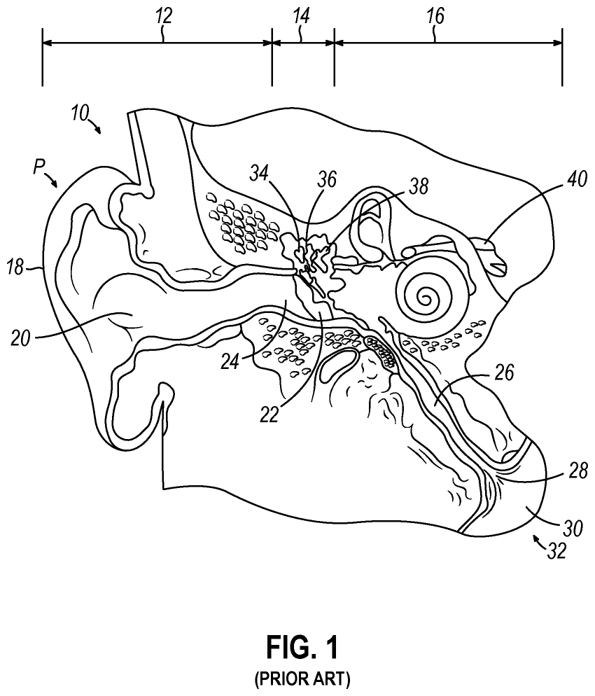

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is, by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Protective Sheaths

A. Exemplary Ear Canal Protective System With First Exemplary Protective Sheath

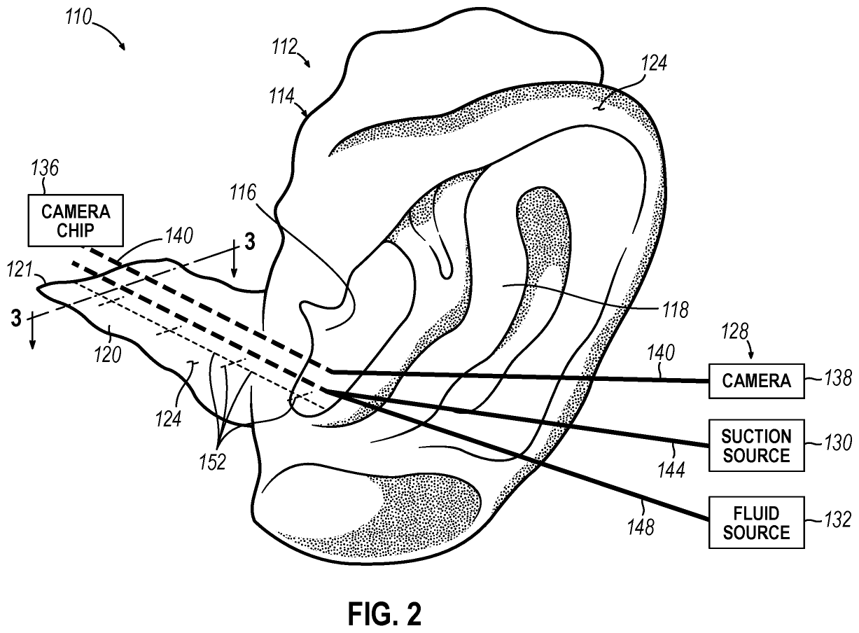

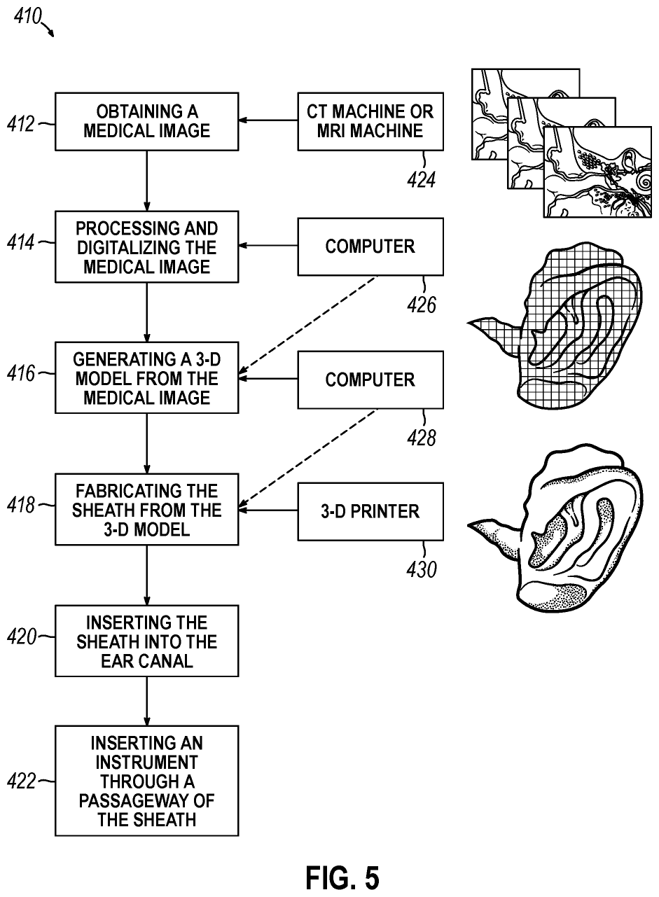

FIG. 2 shows a perspective view of an exemplary ear canal protective system (110) that includes a first exemplary protective sheath (112). Protective sheath (112) is configured to be at least partially inserted into the ear canal (20) of the patient (P). As described in greater detail below with reference to FIG. 5, protective sheath (112) may be custom fabricated, such that protective sheath (112) conforms to the specific surface topography of the ear canal (20) of the patient (P). In other words, it is envisioned that the surface topography of protective sheath (112) would be different for two different patients (i.e. first and second patients). Protective sheath (112) may be formed from a polymeric material (e.g. plastic) or any other suitable material.

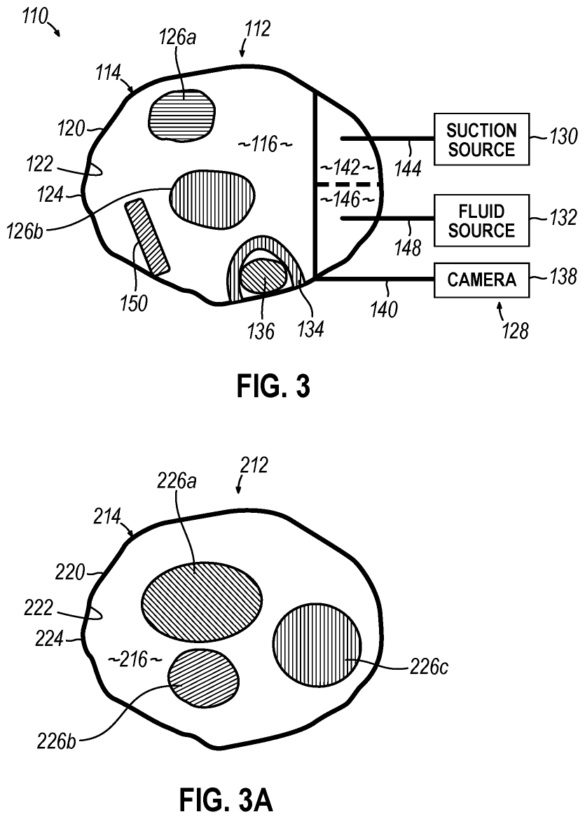

As shown in FIGS. 2-3, protective sheath (112) includes a body (114) and a passageway (116) disposed within body (114). Body (114) includes a proximal portion (118) and a distal portion (120). When positioned within the patient (P), proximal portion (118) may at least partially contact the auricle (18) of the external ear (12) to stabilize protective sheath (112) relative to the ear (10). In the present example, proximal portion (118) is custom-shaped to complement the unique auricle (18) of the particular patient at hand. In some other versions, proximal portion (118) has a different configuration. For instance, some variations may provide a proximal portion (118) that is funnel-shaped, otherwise flared, or otherwise shaped. Regardless, proximal portion (118) may provide a mechanical ground against the external ear (12) to restrict the depth of insertion of distal portion (120) into the ear canal (20).

Distal portion (120) may be at least partially disposed within the ear canal (20). For example, distal portion (120) may extend through a majority of the ear canal (20). More specifically, distal tip (127) of distal portion (120) may terminate within the ear canal (20) at any position prior to contacting the eardrum (22), such that distal tip (127) is laterally spaced away from the eardrum (22).

Body (114) includes an inner surface (122) and an outer surface (124). Inner surface (122) of the body (114) at least partially defines passageway (116). As shown, passageway (116) extends completely through body (114) of protective sheath (112). Body (114) is configured to specifically conform to the shape and dimensions of the ear canal (20) of the patient (P). Body (114) protects the ear canal (20) while inserting at least one instrument (shown as first and second instruments (126a-b) in FIG. 3) through passageway (116) of protective sheath (112). As such, protective sheath (112) minimizes disturbances to the lining and the tissue of the ear canal (20) caused by insertion and subsequent extraction of instruments (126a-b) through passageway (116). These disturbances may otherwise be increased due to repeated insertion and subsequent extraction of instruments (126a-b) through passageway (116) during a surgical procedure. In some versions, outer surface (124) includes a soft outer lining (e.g., a layer of silicone, etc.) to prevent outer surface (124) from damaging the ear canal (20) during insertion and retraction of body (114) through the ear canal (20). Regardless, body (114) may have sufficient rigidity and column strength to enable body (114) to be inserted into the ear canal (20) without buckling.

As shown in FIGS. 2-3, protective sheath (112) is coupled with a camera assembly (128), a suction source (130), and a fluid source (132). FIG. 3 shows a schematic cross-sectional view of protective sheath (112) of FIG. 2, taken along line 3-3 of FIG. 2. As shown in FIG. 3, camera assembly (128) may include an integrated mount (134) fixably coupled to inner surface (122) of body (114). Integrated mount (134) is configured to securably receive a camera chip (136) that is in electrical communication with a camera (138) using a wire (140). Camera chip (136) may be mounted within passageway (116) using integrated mount (134) prior to protective sheath (112) being inserted into the ear (10). Alternatively, camera chip (136) may be mounted within passageway (116) using integrated mount (134) while protective sheath (112) is disposed within the ear canal (20). In some other variations, camera chip (136) is omitted. In some such variations, protective sheath (112) includes a dedicated lumen or other passageway that is configured to receive a conventional endoscope or other visualization instrument.

A portion of passageway (116) may include an integrated suction lumen (142) fluidly coupled with suction source (130) (e.g. a pump) using a tube (144) to remove fluid from the patent (P) during a surgical procedure. A portion of passageway (116) may include an integrated fluid lumen (146) fluidly coupled with fluid source (132) (e.g. a saline solution or another suitable fluid) using a tube (148) to provide fluid to the patient (P) during the surgical procedure. It is envisioned that integrated suction lumen (142) and integrated fluid lumen (146) may be combined into a single lumen, or that integrated suction lumen (142) and/or integrated fluid lumen (146) may be omitted altogether (as shown in FIG. 3A).

Protective sheath (112) of FIG. 3 also includes an integrated irrigation or lens cleaner, shown as a flexible wiper (150), that is disposed within passageway (116). Flexible wiper (150) may be fixably secured to inner surface (122) of body (114). Flexible wiper (150) may be configured to clean a surface of instrument (126a-b) (e.g. an endoscope or another suitable scope). Various other integrated irrigation or lens cleaners are also envisioned.

As shown in FIG. 2, body (114) also includes location marks (152) configured to serve as depth indicia to assist in locating a desired surgical target or determining the longitudinal position of an instrument along the length of distal portion (120). Location marks (152) may be disposed on inner surface (122) and/or outer surface (124). A variety of location markings may be utilized. It is envisioned that body (114) may include none, one, two, three, four, or each of integrated mount (134), integrated suction lumen (142), integrated fluid lumen (146), flexible wiper (150), and location marks (152). Due to its customized design for each patient, protective sheath (112) provides a protective layer over the healthy tissue in the ear canal (20) and minimizes disturbances to this tissue. In addition, due to integrated lumens (142, 146), the movement of instruments (126a-b) inside the ear canal (20) may be reduced.

B. Second Exemplary Protective Sheath

FIG. 3A shows a schematic cross-sectional view of an exemplary alternative protective sheath (212) that is similar to protective sheath (112) of FIG. 3. Similar to protective sheath (112), protective sheath (212) includes a body (214), a passageway (216), a proximal portion (not shown), a distal portion (220), an inner surface (222), and an outer surface (224). Unlike protective sheath (112), protective sheath (212) is not shown as including integrated mount (134), integrated suction lumen (142), integrated fluid lumen (146), flexible wiper (150), or location marks (152). One or more surgical instruments (e.g. first, second, and third instruments (226a-c)) may be inserted through passageway (216) of protective sheath (212). Protective sheath (212) minimizes disturbances to the lining and the tissue of the ear canal (20) caused by insertion and subsequent extraction of instruments (226a-c) through passageway (216).

C. First Exemplary Method of Protecting Ear Canal

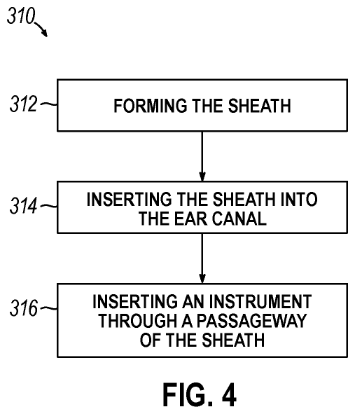

FIG. 4 shows a first exemplary method (310) of protecting the ear canal (20) of the patient (P) using protective sheath (112, 212) of FIGS. 2-3A that includes steps (312, 314, 316). At step (312), method (310) includes forming protective sheath (112, 212) using additive manufacturing. Forming protective sheath (112, 212) using additive manufacturing (e.g. 3-D printing of protective sheath (112, 212)) ensures that the outer contours of protective sheath (112, 212) closely align with unique contours of the interior surface of the patient's ear canal (20). Additionally, forming protective sheath (112, 212) using additive manufacturing may allow for harder and more resilient materials to be used as opposed to a foam or otherwise compressible earpiece. Such hardness and/or resiliency is desirable when one or more instruments (126a-b, 226a-c) are moved within passageway (116, 216). For example, protective sheath (112, 212) formed using additive manufacturing may be non-compressible and already prehardened before being inserted into ear canal (20). For example, as described below with reference to FIG. 5, forming protective sheath (112, 212) may include imaging the ear canal (20), generating a three-dimensional model of the ear canal (20) from the imaging step, and fabricating protective sheath (112, 212) using the three-dimensional model. At step (314), method (310) may include inserting protective sheath (112, 212) into the ear canal (20) of the patient (P), so that passageway (116, 216) extends at least partially within the ear canal (20) of the patient (P) as described above.

At step (316), method (310) also includes inserting at least one instrument (126a-b) through passageway (116, 216) of protective sheath (112, 212) and into the middle ear (14) of the patient (P) during the surgical procedure. For example, instruments (126a-b, 226a-c) may be inserted using a transcanal approach to access the middle ear (14) of the patient (P), where the transcanal approach allows for the eardrum (22) to be endoscopically visualized, and in some cases traversed, via the ear canal (20). During the surgical procedure, method (310) may further include any one or more of removing fluid through integrated suction lumen (142) of passageway (116) and tube (144), providing fluid from fluid source (132) through tube (148) and integrated irrigation lumen (146) of passageway (116), visualizing the surgical procedure using integrated camera chip (136) coupled with camera (138) using wire (140), cleaning a lens or other portion of instrument (126a-b) using a flexible wiper (150) of the integrated lens cleaner coupled with passageway (116) of protective sheath (112), and/or locating a surgical target location using one or more location marks (152) disposed on protective sheath (112). Alternatively, the surgical procedure may utilize protective sheath (212), which is not shown as including any of integrated mount (134), integrated suction lumen (142), integrated fluid lumen (146), flexible wiper (150), or location marks (152).

After removing the instruments (126a-b, 226a-c) from passageway (116, 216), protective sheath (112, 212) may then be removed from the patient (P). It is envisioned that protective sheath (112, 212) may be removed just before completing the surgical procedure. As described above, removing protective sheath (112, 212) from the ear canal (20) after instruments (126a-b, 226a-c) are removed, protects the ear canal (20) from disturbances.

D. Second Exemplary Method of Protecting Ear Canal

FIG. 5 shows a second exemplary method (410) of protecting the ear canal (20) of the patient (P) using protective sheath (112) that includes steps (412, 414, 416, 418, 420, 422). At step (412), method (410) includes imaging the ear canal (20). Imaging of the ear canal (20) may include performing one or more scans of the ear (10) using an imaging machine (424) (e.g. performing a CT scan using a CT machine and/or performing an MRI scan using an MRI machine). Computed tomography (CT) is a diagnostic imaging test used to generate detailed images of internal organs, bones, soft tissue and blood vessels of the patient (P). Magnetic resonance imaging (MRI) is a technique that uses a magnetic field and radio waves to create detailed images of the organs and tissues within the patient (P). Other imaging techniques are also envisioned, including but not limited to optical scanning from within the ear canal (20). Preoperative testing (e.g. prior to the surgical procedure) may assist in generating the imaging data of the ear (10). The imaging may correspond to only a portion of the ear (10) (e.g. as the ear canal (20)), or alternatively, the imaging may correspond to the entirety of the ear (10) (e.g., including the auricle (18) of the external ear (12)).

At step (414), method (410) includes processing and digitalizing the medical image(s) obtained from imaging machine (424) using a computer (426). For example, processing and digitalizing the medical image(s) may include stitching together discrete images to form a customized surface topography of the ear canal (20). At step (416), method (410) includes generating a three-dimensional digital model of the ear canal (20) using the customized surface topography of the ear canal (20) from step (414) using a computer (428). By way of example only, the final three-dimensional model may be in the form of an STL digital file, or another suitable digital file. It is envisioned that computer (426) may be the same or different than computer (428). It is also envisioned that steps (414, 416) may be combined into a single step. For example, once the image(s) of the ear canal (20) have been obtained, a processor (not shown), but part of computer (426, 428) may synthesize this medical image data to form the three-dimensional model. In some versions, the three-dimensional digital model of the ear canal (20) is stitched together with a generic grounding structure (e.g., funnel-shaped structure) that will constitute the proximal portion (118) of the sheath (112). In some other versions, the three-dimensional digital model includes the auricle (18) of the external ear (12); in addition to including the ear canal (20) of the patient at hand.

The three-dimensional model may be used in numerous ways. As another merely illustrative example, a three-dimensional model that has been generated may be combined with other preoperative image data (e.g., CT scans, MRI scans, etc.) to create a composite three-dimensional model or to otherwise refine the three-dimensional model. Various suitable ways in which a composite three-dimensional model may be and other preoperative image data (e.g., CT scans, MRI scans, etc.) will be apparent to those skilled in the art in view of the teachings herein. By way of example only, such data may be combined to generate a composite three-dimensional model in accordance with at least some of the teachings of U.S. Pat. No. 8,199,988, entitled “Method and Apparatus for Combining 3D Dental Scans with Other 3D Data Sets,” issued Jun. 12, 2012, the disclosure of which is incorporated by reference herein; and/or “U.S. Pat. No. 8,821,158, entitled “Method and Apparatus for Matching Digital Three-Dimensional Dental Models with Digital Three-Dimensional Cranio-Facial CAT Scan Records,” issued Sep. 2, 2014, the disclosure of which is incorporated by reference herein.

At step (418), method (410) includes fabricating protective sheath (112, 212) using the three-dimensional model (or the composite three-dimensional model). For example, fabricating protective sheath (112, 212) may include forming protective sheath (112, 212) using additive manufacturing using the three-dimensional model. More specifically, a 3-D printer (430) may fabricate protective sheath (112, 212). As described above, protective sheath (112, 212) includes passageway (116, 216) that extends through body (114, 214). In versions where the three-dimensional digital model includes a digital representation of the auricle (18) of the patient, the fabrication step (418) may include fabricating proximal portion (118) as a hollow version of the auricle (18) to thereby allow the auricle (18) of the patient to be inserted into proximal portion (118) when protective sheath (112, 212) is fitted to the patient as described below. Alternatively, the fabrication step (418) may include fabricating proximal portion (118) as a generic structure (e.g., a funnel-like structure) that is not necessarily specific to the patient at hand. In either case, the fabrication step (418) may nevertheless include fabricating distal portion (120) as a complement to the ear canal (20) of the particular patient at hand, such that outer surface (124, 224) conforms to the unique contours of the interior surface of the patient's ear canal (20).

Forming protective sheath (112, 212) using additive manufacturing (e.g. 3-D printing of protective sheath (112, 212)) ensures that the outer contours of protective sheath (112, 212) closely align with the unique contours of the interior surface of the patient's ear canal (20). Additionally, forming protective sheath (112, 212) using additive manufacturing may allow for harder and more resilient materials to be used as opposed to a foam or otherwise compressible earpiece. Such hardness and/or resiliency is desirable when one or more instruments (126a-b, 226a-c) are moved within passageway (116, 216). For example, protective sheath (112, 212) formed using additive manufacturing may be non-compressible and already pre-hardened before being inserted into ear canal (20).

At step (420), method (410) includes inserting protective sheath (112, 212) into the ear canal (20) of the patient (P), so that passageway (116, 216) extends at least partially within the ear canal (20) of the patient (P). As noted above, regardless of whether the proximal portion (118) is configured like a hollow version of the auricle (18) of the external ear (12) of the patient at hand, or the proximal portion (118) has some other kind of structure (e.g., a generic funnel-like structure, etc.), the proximal portion (118) may arrest movement of protective sheath (112, 212) during the insertion step (420), thereby preventing distal portion (120) from inadvertently contacting the eardrum (22) of the patient.

At step (422), method (410) includes inserting at least one instrument (126a-b, 226a-c) during the surgical procedure through passageway (116, 216) of protective sheath (112, 212) and into the middle ear (14) of the patient (P). During the surgical procedure, method (410) may further include any one or more of removing fluid through integrated suction lumen (142) of passageway (116) and tube (144), providing fluid from fluid source (132) through tube (148) and through integrated irrigation lumen (146) of passageway (116), visualizing the surgical procedure using integrated camera chip (136) coupled with camera (138) using wire (140), cleaning a lens of instrument (126a-b) using a flexible wiper (150) of the integrated lens cleaner, and/or locating a surgical target location using one or more location marks (152) disposed on protective sheath (112). Alternatively, the surgical procedure may utilize protective sheath (212), which is not shown as including integrated mount (134), integrated suction lumen (142), integrated fluid lumen (146), flexible wiper (150), or location marks (152). In some procedures, the at least one instrument (126a-b, 226a-c) stops short of the eardrum (22). In some other versions, at least one instrument (126a-b, 226a-c) traverses the eardrum (22) and enters the inner ear (16). In some such procedures, the instrument includes a dilation catheter that is further inserted into the ET (26) via the inner ear (16), to thereby dilate the ET (26). As shown, protective sheaths (112, 212) are not part of a hearing aid, and as a result, do not include a microphone, a processor or a receiver.

After removing the surgical instrument(s) (e.g. instruments (126a-b, 226a-c)) from passageway (116, 216), protective sheath (112, 212) may then be removed from the patient (P). It is envisioned that protective sheath (112, 212) may be removed just before completing the surgical procedure. As described above, removing protective sheath (112, 212) from the ear canal (20) after instruments (126a-b, 226a-c) are removed, protects the ear canal (20) from potential damage. Due to its customized design for each individual patient, protective sheath (112, 212) provides a close-fitting protective layer over the healthy tissue in the ear canal (20) and minimizes disturbances to this tissue in the ear canal (20). In addition, due to integrated lumens (142, 146), the movement of instruments (126a-b) inside the ear canal (20) may be reduced, offering additional benefits for protective sheath (112).

II. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A method of protecting an ear canal of a patient during a surgical procedure using a protective sheath, the method comprising: (a) forming the protective sheath using additive manufacturing based on a digital model of anatomy of the patient, wherein the protective sheath includes a passageway extending therethrough; (b)inserting the protective sheath into the patient so that the passageway extends at least partially within the ear canal of the patient; and (c) inserting at least one instrument during the surgical procedure through the passageway of the protective sheath and into the middle ear of the patient.

### EXAMPLE 2

The method of Example 1, wherein forming the protective sheath further comprises custom fabricating the protective sheath for the specific surface topography of the ear canal of the patient.

### EXAMPLE 3

The method of any one or more of Examples 1 through 2, wherein the protective sheath is not part of a hearing aid.

### EXAMPLE 4

The method of any one or more of Examples 1 through 3, further comprising removing the protective sheath from the patient during the surgical procedure after removing the at least one instrument from the passageway.

### EXAMPLE

The method of any one or more of Examples 1 through 4, further comprising protecting the ear canal using the protective sheath while inserting the at least one instrument through the passageway of the protective sheath.

### EXAMPLE 6

The method of any one or more of Examples 1 through 5, wherein the protective sheath minimizes disturbances to a lining and the tissue of the ear canal caused by insertion and subsequent extraction of the at least one instrument through the passageway.

### EXAMPLE 7

The method of any one or more of Examples 1 through 6, further comprising inserting the at least one instrument using a transcanal approach to access the middle ear of the patient, wherein the transcanal approach allows for the eardrum to be endoscopically visualized and accessed through the ear canal.

### EXAMPLE 8

The method of any one or more of Examples 1 through 7, wherein forming the protective sheath further includes: (i) imaging the ear canal, (ii) generating the digital model to be a three-dimensional digital model of the ear canal from the imaging step, and (iii) fabricating the protective sheath using the three-dimensional model.

### EXAMPLE 9

The method of Example 8, wherein imaging the ear canal further comprises performing a CT scan or an MRI scan of the ear canal that is used to create the three-dimensional model.

### EXAMPLE 10

The method of any one or more of Examples 8 through 9, herein forming the protective sheath further comprises forming the protective sheath using additive manufacturing using the three-dimensional model.

### EXAMPLE b 11

The method of any one or more of Examples 1 through 10, further comprising removing fluid through an integrated suction lumen of the passageway.

### EXAMPLE 12

The method of any one or more of Examples 1 through 11, further comprising providing fluid from a fluid source through an integrated irrigation lumen of the passageway.

### EXAMPLE 13

The method of any one or more of Examples 1 through 12, further comprising visualizing the surgical procedure using an integrated camera chip coupled with a camera using a wire.

### EXAMPLE 14

The method of any one or more of Examples 1 through 13, further comprising cleaning a lens of an endoscope using an integrated lens cleaner coupled with the passageway of the protective sheath.

### EXAMPLE 15

The method of any one or more of Examples 1 through 14, further comprising locating a surgical target location using one or more location marks disposed on the protective sheath.

### EXAMPLE 16

A method of protecting an ear canal of a patient using a protective sheath, the method comprising: (a) imaging the ear canal of the patient; (b) generating a three-dimensional model of the ear canal from the imaging step; (c) fabricating the protective sheath using the three-dimensional model, wherein the protective sheath includes a passageway extending therethrough; (d) inserting the protective sheath into the patient so that the passageway extends at least partially within the ear canal of the patient; and (e) inserting at least one instrument during the surgical procedure through the passageway of the protective sheath and into the middle ear of the patient.

### EXAMPLE 17

The method of any one or more of Examples 8, 10, or 16, wherein fabricating the protective sheath further comprises forming the protective sheath using additive manufacturing using the three-dimensional model.

### EXAMPLE 18

The method of any one or more of Examples 8, 9, 16 or 17, wherein imaging the ear canal further comprises performing a CT scan or an MRI scan.

### EXAMPLE 19

A protective sheath configured to be inserted into the ear canal of a patient, wherein the protective sheath comprises: (a) a body configured to specifically contour to the shape and dimensions of the ear canal of the patient; and (b) a passageway extending completely through the body of the protective sheath, wherein the body includes one or more of an integrated mount configured to receive a camera chip, an integrated irrigation lens cleaner.

### EXAMPLE 20

The method of Example 19, wherein the body includes each of the integrated mount configured to receive the camera chip, the integrated lens cleaner, or location marks configured to locate a desired surgical target.

III. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, examples, etc. described herein may be combined with any one or more of the other teachings, expressions, examples, etc. that are described herein. The above-described teachings, expressions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various examples of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method of protecting an ear canal of a patient during a surgical procedure using a protective sheath, the method comprising:
(a) forming the protective sheath using additive manufacturing based on a digital model of anatomy of the patient, wherein the protective sheath includes a passageway extending therethrough;
(b) inserting the protective sheath into the patient so that the passageway extends at least partially within the ear canal of the patient; and
(c) inserting at least one instrument during the surgical procedure through the passageway of the protective sheath and into the middle ear of the patient.

2. The method of claim 1, wherein forming the protective sheath further comprises custom fabricating the protective sheath for the specific surface topography of the ear canal of the patient.

3. The method of claim 1, wherein the protective sheath is not part of a hearing aid.

4. The method of claim 1, further comprising removing the protective sheath from the patient during the surgical procedure after removing the at least one instrument from the passageway.

5. The method of claim 1, further comprising protecting the ear canal using the protective sheath while inserting the at least one instrument through the passageway of the protective sheath.

6. The method of claim 1, wherein the protective sheath minimizes disturbances to a lining and the tissue of the ear canal caused by insertion and subsequent extraction of the at least one instrument through the passageway.

7. The method of claim 1, further comprising inserting the at least one instrument using a transcanal approach to access the middle ear of the patient, wherein the transcanal approach allows for the eardrum to be endoscopically visualized and accessed through the ear canal.

8. The method of claim 1, wherein forming the protective sheath further includes:
(i) imaging the ear canal of the patient,
(ii) generating the digital model to be a three-dimensional digital model of the ear canal from the imaging step, and
(iii) fabricating the protective sheath using the three-dimensional model.

9. The method of claim 8, wherein imaging the ear canal further comprises performing a CT scan or an MRI scan of the ear canal that is used to create the three-dimensional model.

10. The method of claim 8, wherein forming the protective sheath further comprises forming the protective sheath using additive manufacturing using the three-dimensional model.

11. The method of claim 1, further comprising removing fluid through an integrated suction lumen of the passageway.

12. The method of claim 1, further comprising providing fluid from a fluid source through an integrated irrigation lumen of the passageway.

13. The method of claim 1, further comprising visualizing the surgical procedure using an integrated camera chip coupled with a camera using a wire.

14. The method of claim 1, further comprising cleaning a lens of an endoscope using an integrated lens cleaner coupled with the passageway of the protective sheath.

15. The method of claim 1, further comprising locating a surgical target location using one or more location marks disposed on the protective sheath.

16. A method of protecting an ear canal of a patient using a protective sheath, the method comprising:
(a) imaging the ear canal of the patient;
(b) generating a three-dimensional model of the ear canal from the imaging step;
(c) fabricating the protective sheath using the three-dimensional model, wherein the protective sheath includes a passageway extending therethrough;
(d) inserting the protective sheath into the patient so that the passageway extends at least partially within the ear canal of the patient; and
(e) inserting at least one instrument during the surgical procedure through the passageway of the protective sheath and into the middle ear of the patient.

17. The method of claim 16, wherein fabricating the protective sheath further comprises forming the protective sheath using additive manufacturing using the three-dimensional model.

18. The method of claim 16, wherein imaging the ear canal further comprises performing a CT scan or an MM scan.

19. A protective sheath configured to be inserted into the ear canal of a patient, wherein the protective sheath comprises:
(a) a body configured to specifically contour to the shape and dimensions of the ear canal of the patient; and
(b) a passageway extending completely through the body of the protective sheath, wherein the body includes one or more of an integrated mount configured to receive a camera chip, an integrated irrigation lens cleaner.

20. The protective sheath of claim 19, wherein the body includes each of the integrated mount configured to receive the camera chip, the integrated lens cleaner, or location marks configured to locate a desired surgical target.
